(19) United States
(12) Patent Application Publication
(10) Pub. No.: US 2025/0104298 A1
(43) Pub. Date: Mar. 27, 2025

(54) METHOD OF HARMONIZING AN MR IMAGE USING A FLOW MODEL AND AN MRI APPARATUS FOR THE SAME

(71) Applicant: SEOUL NATIONAL UNIVERSITY R&DB FOUNDATION, Seoul (KR)

(72) Inventors: Jongho LEE, Seoul (KR); Hwihun JEONG, Seoul (KR)

(21) Appl. No.: 18/423,435

(22) Filed: Jan. 26, 2024

(30) Foreign Application Priority Data
Sep. 27, 2023 (KR) 10-2023-0130180

(51) Int. Cl. G06T 11/00; G06T 7/30; G06T 7/70

(52) U.S. Cl. CPC G06T 11/006; G06T 7/30; G06T 7/70; G06T 2207/10088; G06T 2207/20081; G06T 2207/30016; G06T 2207/30096; G06T 2210/41

(57) ABSTRACT

An MR image processing method that repeats, by a computing device, a unit transformation method N number of times is provided. The unit transformation method in n^{th} iteration comprises, generating a corrected image corrected from a prepared n^{th} image using a predetermined reversible generative model, calculating a differential value of a distance between the corrected image and a source image, and generating a harmonized image by subtracting the differential value from the corrected image. The generating the corrected image comprises, generating a predetermined array by inputting the n^{th} image into the reversible generative model in a forward direction of the reversible generative model, and generating the corrected image by inputting a scaled array in a reverse direction of the reversible generative model, the scaled array being obtained by scaling a value of each element of the generated array by a predetermined scaling factor (1−α) (0<α<1).

### CROSS-REFERENCE TO RELATED APPLICATION

This application claims priority to Korean Patent Application No. 10-2023-0130180 filed on Sep. 27, 2023, and all the benefits accruing therefrom under 35 U.S.C. § 119, the contents of which are incorporated by reference in their entirety.

### BACKGROUND

The present invention relates to MRI image processing, and in particular, to a technology for transforming an MR source image obtained from a source domain into a harmonic image for a target domain.

Magnetic resonance imaging (MRI) is a widely-used medical imaging modality. With the advent of deep learning-powered computer vision techniques, there have been numerous applications of deep learning in MRI, such as disease classification, tumor segmentation, and solving inverse problems. Despite the notable performance of deep learning in MRI, its widespread usage has been hindered by the inherent domain gap present in MRI data. Variations occur in MRI images across different vendors, scanners, sites, and scan parameters. This domain gap presents a generalization issue when applying the data to a neural network that has been trained on a different dataset.

To overcome the challenges of generalization in deep learning applied to MRI data, several harmonization methods have been developed to match the source domain image to the characteristics of the target domain. These approaches include non-deep learning-based methods and deep learning-based methods, which have demonstrated significant performance improvements. However, there are limitations that need to be addressed in these methods. Firstly, many of these methods require multiple datasets from different domains. For instance, DeepHarmony which is a supervised end-to-end framework requires “traveling subjects” who undergo multiple MRI scans with different scanners to obtain images from both the source and target domains. The application of CycleGAN-based style transfer can mitigate the need for traveling subjects (=scan object), it still necessitates large datasets with multiple domains. Secondly, the harmonization network trained for mapping between specific source and target domains is challenging to be applied in novel domains, limiting the generalizability of methods. Efforts have been made to adopt a disentanglement approach and domain adaptation to perform harmonization in unseen domains, but it requires a multi-contrast and multi-site paired dataset.

In recent years, a class of invertible generative models called normalizing flows has been introduced and has shown exceptional performance in a broad range of computer vision tasks. Normalizing flows have a unique capability of not only generating novel images that resemble samples from the distribution of a specific dataset but also mapping the probabilistic distribution of image datasets. This feature makes normalizing flows particularly well-suited for image generation and manipulation tasks, as they can effectively capture the underlying distribution of image data and generate new images that are consistent with that distribution. Furthermore, the invertibility of these models provides fine-grained control over the generated images, making them useful for tasks such as image editing and style transfer.

### SUMMARY

The present invention aims to provide the concept of “Blind Harmonization”, where the harmonization network can be constructed only with the target domain data during training and applicable to diverse source domains that are unseen during training.

The present invention aims to provide a technique obtaining harmonized data (a harmonized image) from source image data (a source domain image) using the Blind Harmonization concept.

The present invention aims to find the harmonized image that sustains the anatomical structure and contrast of the input image while maintaining a high likelihood in the flow model (i.e., enabling harmonization for the target domain), leveraging the invertibility of flow models.

According to one aspect of the present invention, a method is provided for constructing a harmonization network using only target domain data, and obtaining a harmonized data from data of a source domain using the harmonization network. The method can be called as a Blind Harmonization in the specification.

### <Mri Harmonization>

There are numerous demands for harmonizing MR images from different sites, vendors, or scanners. Several studies have proposed methods for harmonizing MR images from the source domain to the target domain. Conventionally, these approaches have relied on post-processing techniques at the image level, such as histogram matching and statistical normalization, which aims to adjust the intensity values of the images to make them more similar. However, with recent advances in deep learning, there has been growing interest in developing deep learning-based methods for harmonization. One popular deep learning-based method is DeepHarmony, which utilizes an end-to-end supervised framework to learn the mapping between the source and target domains. Although DeepHarmony has demonstrated promising results, it requires a large dataset of traveling subjects for training, which can be difficult to acquire. To address this limitation, researchers have employed CycleGAN-based style transfer networks. CycleGAN is a generative adversarial network (GAN) that can learn to map images from one domain to another without the need for paired data. By training CycleGAN on a large dataset of MR images, it is possible to generate images that are visually similar to the target domain while retaining the relevant anatomical features. More recently, researchers have developed separated networks for the contrast network and structure network to enable more flexible applicability. The technique called CALAMITI is a GAN-based method that disentangles the contrast and structural information in MR images and allows for more granular control over the image properties that need to be harmonized. In addition to image-level transformations, some works have focused on feature-level harmonization. These methods aim to learn a common feature representation that can be used for downstream analysis tasks. For example, task-based harmonization methods learn a task specific feature representation that can improve the performance of a specific analysis task. In this specification, the term ‘MR image’ can be called as ‘MRI image’.

### <Normalizing Flow>

The flow-based model is a family of generative models, known as normalizing flows, that enables the parametrization of complex data distributions with a series of invertible transformations from simple random variables. Normalizing flows were first introduced, and the so-called NICE model was proposed as a deep learning framework that maps the complex high-dimensional density of training data to a simple factorized space using non-linear bijective transformations. Substantial improvements in invertible neural networks and high-quality image generation from the sample space have been achieved. In particular, the technique called as GLOW proposed an efficient and parallelizable transformation using invertible Ix 1 convolutions for designing invertible neural networks and demonstrated remarkable results in high-resolution image synthesis tasks. By introducing a log-likelihood-based model in normalizing flows, GLOW can efficiently generate high-resolution natural images. Recent studies have demonstrated great performance of the normalizing flows model in a wide range of computer vision tasks such as super-resolution, denoising, and colorization, using the properties of the normalizing flows model. Among them, the technique called as SRFlows adopted negative log-likelihood loss and successfully generated more diverse super-resolution images than GAN-based approaches by conditioning on low-resolution images.

Here, the reversible neural network may refer to a neural network in which there is a one-to-one correspondence between the input and output of the neural network.

### <Prior-Based Optimization>

Conventionally, the inverse problem of y=Ax is widely solved by using regularization techniques. With the advent of generative models, several studies have proposed methods that solve this problem using generative models as prior models or regularizers. Generative adversarial networks (GANs), flow models, and deep image priors are commonly used as priors. Individual training of these priors has the benefit of generalizability in the matrix A. For example, in the case of reconstructing MR images from undersampled images, using a generative model prior can be applied to diverse sampling masks.

Here, the ‘prior’ may mean a previously known characteristic or probability of x.

### <Harmonization Model>

When a subject undergoes multiple scans with different vendors or MRI scan parameters, the resulting MR images exhibit differences, mainly in low-frequency, while the structural differences are relatively small. This provides some insight into the relationship between images from different domains. Firstly, the images are highly correlated, as the difference between domains does not significantly impact the overall. Secondly, the edges of the images coincide. Given xₛ as the source domain image and xₕ as its corresponding harmonized version to the target domain, the following equation 1 and equation 2 hold due to the correlation and edge coincidence.

$$NCC ( {x}_{h} , {x}_{s} ) \approx 1 , \qquad [ Equation 1 ]$$

$$\vert {MGx}_{h} \vert \approx 0 \qquad [ Equation 2 ]$$

In these equations, M is a mask obtained by thresholding the gradient value of xₛ, which retains only the non-edge regions. G represents the gradient operator, and NCC denotes normalized cross-correlation. Equation 1 suggests that the harmonized image should have a high crosscorrelation value with the source domain image. Equation 2 enforces edge sparsity in the harmonized image within regions where the source domain image is considered not to have any edges.

Based on Equation and Equation 2, it can be defined a distance measure, D, between the source domain image and the harmonized image, as shown in equation 3.

$$D ( {x}_{h} , {x}_{s} ) = {β}_{1} \{ 1 - NCC ( {x}_{h} , {x}_{S} ) \} + {β}_{2} M \vert {Gx}_{h} \vert \qquad [ Equation 3 ]$$

Here, βₛ are hyperparameters. However, the problem of finding xₕ that satisfies D(xₕ, xₛ)=0 given xₛ is highly ill-posed, as there exists a trivial solution of xₕ=xₛ. Nevertheless, if the prior distribution of the target domain p_{X}(x) is given, the problem can be solved using a regularization approach as shown in Equation 4.

$${x}_{h}^{″} = arg {min}_{x} D ( x , {x}_{s} ) - α log {p}_{X} ( x ) \qquad [ Equation 4 ]$$

Here, a is a regularization parameter. The remaining issue is how to estimate the prior distribution of the target domain and optimize the solution for x″ₕ. According to the present invention, the normalizing flow model has been selected to map the distribution of the target domain, as the inherent invertibility of the flow model can provide an advantage for optimization.

Here, the prior distribution may mean a unique distribution of the image.

### <Flow-Based Prior Learning>

A normalizing flow is an invertible transformation that maps a sample from a simple probability distribution (such as a normal Gaussian) to a sample from a complex probability distribution. The transformation itself (which is often called “flow”) and its inverse are assumed to be differentiable.

Let ZΣR^{D} be a random variable with an associated probability density function (PDF) p_{Z}: R^{D}→[0, 1] which is assumed to be known and tractable. Let f_{θ}: R^{D}→R^{D} be a diffeomorphism parameterized by the vector θ∈R^{P} with an inverse denoted by f_{θ}⁻¹. Then the PDF of the random variable X=f_{θ}⁻¹(Z) can be computed explicitly using the change of variables formula as shown in Equation 5.

$${p}_{X} ( x \vert θ ) = {p}_{Z} ( {f}_{θ} ( x ) ) \vert det ( \partial {f}_{θ} ( x ) ) / \partial x ) \vert \qquad [ Equation 5 ]$$

Here, ∂f_{θ}(x))/∂x is the Jacobian of f_{θ}.

When applying normalizing flows for sample generation or density estimation problems, the simple distribution p_{Z}, known as the “latent distribution”, is transformed via the “flow” f_{θ} to a more complex distribution p_{X}. The objective for both problems is to find the value of the parameters θ for which p_{X} closely approximates the underlying distribution p_{data} of the given dataset. Only after the objective is satisfied can we accurately estimate densities of random samples using the change of variable formula or generate random samples that are consistent with the given data by first sampling from the latent distribution and f_{feeding} the sample to the inverse of the flow.

The aforementioned objective can be stated formally as a maximum likelihood estimation (MLE) problem: maximizing the expected log-likelihood over the possible values of the parameters θ∈R^{P}, as shown in Equation 6, Equation 7, and Equation 8, where D:={x⁽ⁱ⁾}^{N}ᵢ₌₁ is a given dataset.

$$L ( θ ; x ) := {E}_{X \sim pdata} [ log ( {p}_{X} ( X \vert θ ) ) ] \qquad [ Equation 6 ]$$

$$\approx ( 1 / N ) {\sum }_{from i = 1}^{to i = N} log ( {p}_{X} ( {x}^{( i )} \vert θ ) ) \qquad [ Equation 7 ]$$

$$\approx ( 1 / N ) {\sum }_{from i = 1}^{to i = N} [ log ( {p}_{Z} ( {f}_{θ} ( {x}^{( i )} ) ) ) + log \vert det ( \partial {f}_{θ} ( {x}^{( i )} ) / \partial x ) \vert ] \qquad [ Equation 8 ]$$

Therefore, training the normalizing flow involves updating the parameters of the flow so that the expected log-likelihood is maximized.

In order to accurately and efficiently approximate the target distribution p_{data}, a normalizing flow f_{θ} must satisfy several conditions: It must be a bijection with differentiable forward and inverse transformations, it must be expressive enough to model the complexity of the target distribution, and the computations of f_{θ}, f_{θ}⁻¹, and det(∂f_{θ}(x)/∂x) must be done efficiently.

Therefore, many state-of-the-art normalizing flows use neural networks that are carefully designed to have differentiable inverse transformations and a Jacobian matrix whose determinant can be calculated efficiently. These include coupling transforms, which have been shown to be particularly effective.

### <BlindHarmony Optimization>

According to one aspect of the present invention, in order to harmonize images from unknown domains, an unconditional flow model is trained solely on the target domain. The prior distribution of the harmonized image x can be parameterized as following Equation 9.

$$log ( {p}_{X} ( x ) ) = log ( {p}_{Z} ( z ) + log \vert det ( \partial {f}_{θ} ( x ) / \partial x ) \vert \qquad [ Equation 9 ]$$

If z is a normal Gaussian, Equation 4 in the z-domain can be re-written as following Equation 10, and Equation 11.

$${z}_{h}^{″} = arg {min}_{z} D ( {f}_{θ}^{- 1} ( z ) , {x}_{s} ) + α log {p}_{Z} ( z ) \qquad [ Equation 10 ]$$

$${z}_{h}^{″} = arg {min}_{z} D ( {f}_{θ}^{- 1} ( z ) , {x}_{s} ) + α {\vert z \vert}^{2} \qquad [ Equation 11 ]$$

The optimization process of Equation 11 (Equation 10) requires the calculation of gradients of f_{θ}−¹(z), which can be computationally burdensome. To increase computational efficiency and reduce processing time, the calculation of ∂f_{θ}⁻¹(z)/∂z. simply can be dropped. Instead, iterative optimization is performed in both the z- and x-domains, by leveraging the invertibility of normalizing flow. The algorithm alternates between taking a gradient descent of the distance measure D(x, xₛ) and the prior term |z|².

In the latent vector domain z, z is updated so that it does not deviate far from the center of the Gaussian as shown in Equation 12.

$$z \to ( 1 - α ) z \qquad [ Equation 12 ]$$

In the image domain x, the gradient of D(x, xₛ) is measured and updated at each iteration as following Equation 13.

$$x \to x + {β}_{1} {∇}_{x} NCC ( x , {x}_{s} ) - {β}_{2} {∇}_{x} \vert MGx \vert \qquad [ Equation 13 ]$$

After N iterations, the resultant image x is the harmonized image x″ₕ.

A BlindHarmony optimization method according to one aspect of the present invention is formulated as Algorithm 1 and FIG. 5. The hyperparameters α, β₁, and β₂ are found heuristically using grid search. Hyperparameters were fixed as: α=0.001, β₁=1000, β₂=0.001, N=10. The initial image x₁ is selected to be an averaged image of training data image.

Algorithm 1 above is presented as follows.

In step S410, a source domain image xₛ, a harmonized image x″ₕ, the flow model f_{θ} learned in a target domain, an initial image x₀, hyperparameters α, β₁, and β₂, and the number of iterations N may be prepared in advance.

In step S420, xₙ₊₁ is calculated according to Equation 14.

$${x}_{n + 1} = {f}_{θ}^{- 1} ( {z}_{n} ) + {β}_{1} {∇}_{x} NCC ( {f}_{θ}^{- 1} ( {z}_{n} ) , {x}_{s} ) - {β}_{2} {∇}_{x} \vert {MGf}_{θ}^{- 1} ( {z}_{n} ) \vert \qquad [ Equation 14 ]$$

In step S430, zₙ₊₁ is calculated according to Equation 15.

$${z}_{n + 1} = ( 1 - α ) {f}_{θ}^{- 1} ( {x}_{n + 1} ) \qquad [ Equation 15 ]$$

The steps S420 and S430 are repeated N times while increasing n by 0 from 1 to N−1.

After the steps S420 and S430 are repeated N times, x_{N} obtained is determined to be the harmonized image x″ₕ of the source domain image xₛ.

According to one aspect of the present invention, as a solution for blind harmonization, a flow-based blind MR image harmonization framework that utilizes only target domain information in the learning process may be provided. This framework may be referred to herein as BlindHarmony.

The blind harmonization method provided according to one aspect of the present invention is intended to solve the limitations of existing harmonization models, which generally require multiple datasets during training or deteriorate performance when applied to datasets from unseen domains. By learning a model with only target domain data, blind harmonization can generalize to unseen source domains. The blind harmonization method provided according to one aspect of the present invention is a new method that improves the generalizability of harmonization methods in medical imaging.

A blind harmonization framework provided in accordance with one aspect of the present invention operates in the following manner. Initially, a flow model is trained only on target domain data to estimate a prior distribution of the target domain. Afterwards, harmonization from the source domain to the target domain is performed iteratively using the trained flow model. The length of a latent variable z is reduced by (1−α), making z closer to the Gaussian center. Gradient descent is used to ensure that the generated image is not too far from the source domain image.

According to one aspect of the present invention, An MR image processing method that repeats, by a computing device, a unit transformation method N number of times can be provided. The unit transformation method in n^{th} iteration comprises: generating, by the computing device, a corrected image corrected from a prepared n^{th} image using a predetermined reversible generative model; calculating, by the computing device, a differential value of a distance between the corrected image and a source image; and generating, by the computing device, a harmonized image by subtracting the differential value from the corrected image. Here, the generating the corrected image comprises: generating a predetermined array by inputting the n^{th} image into the reversible generative model in a forward direction of the reversible generative model; and generating the corrected image by inputting a scaled array in a reverse direction of the reversible generative model, the scaled array being obtained by scaling a value of each element of the generated array by a predetermined scaling factor (1−α) (0<α<1).

The unit transformation method is for transforming a source image into a harmonized image. In this specification, the term ‘unit transformation method’ can be called as ‘unit conversion method’.

Here, information output along the reverse direction of the reversible generative model may be the corrected image.

Here, the reversible generative model may be a prior model.

Here, the n^{th} image (xₙ) input to the reversible generative model (f_{θ}) in the n^{th} iteration of the unit transformation method may be the harmonized image (xₙ) generated in (n−1)^{th} iteration of the unit transformation method (n=2, . . . , N).

Here, the reversible generative model (f_{θ}) may be learned using only images belonging to a first domain.

Here, in a first iteration (n=1) of the unit transformation method, a first image (x₁) input to the reversible generative model (f_{θ}) may be either a random image or one of images belonging to the first domain, or a representative image of images belonging to the first domain.

Here, the source image (xₛ) may be an image belonging to a second domain different from the first domain.

Here, the MR image processing method may further comprise: inputting, by the computing device, a harmonized image (x_{N+1}) generated by the unit transformation method performed by the n^{th} iteration into a predetermined estimation network learned using images belonging to the first domain; and obtaining, by the computing device, an estimate from the estimation network according to the inputted harmonized image (x_{N+1}).

Here, the source image may be a first MRI image of a brain, and the estimation network may be a network that outputs a location of a tumor among the first MRI image of the brain when the first MRI image of the brain is input to the estimation network.

Here, the source image may be an MRI image of a human body, and the estimation network may be a network that outputs a location of a lesion in the MRI image of the human body when the MRI image of the human body is input to the estimation network.

Here, the first domain may be a domain composed of images output by a first MR scanner, and the second domain may be a domain composed of images output by a second MR scanner. In this specification, the term ‘MR scanner’ can be called as ‘MRI scanner’.

Here, a method for learning the reversible generative model (f_{θ}) may comprise: inputting a selected image belonging to the first domain into the reversible generative model in the forward direction of the reversible generative model to generate an array from the reversible generative model; and changing parameters of the reversible generative model to reduce a difference between distributions of images belonging to the first domain and the generated array.

According another aspect of the present invention, an MR image processing method can be provided. The MR image processing method comprises: generating, by an MRI scanner, a source image (xₛ); and repeating, by a computing device, a unit transformation method N number of times, the unit transformation method being a method to transform the generated source image (xₛ) into a harmonized image. Here, the unit transformation method in n^{th} iteration comprises: generating, by the computing device, a corrected image corrected from a prepared n^{th} image using a predetermined reversible generative model; calculating, by the computing device, a differential value of a distance between the corrected image and a source image; and generating, by the computing device, a harmonized image by subtracting the differential value from the corrected image. Here, the generating the corrected image comprises: generating a predetermined array by inputting the n^{th} image into the reversible generative model in a forward direction of the reversible generative model; and generating the corrected image by inputting a scaled array in a reverse direction of the reversible generative model, the scaled array being obtained by scaling a value of each element of the generated array by a predetermined scaling factor (1−α) (0<α<1).

According to still another aspect of the present invention, an MR image processing system comprising an MRI scanner; and a computing device can be provided. Here, the MRI scanner is configured to scan a scan object to generate a source image (xₛ), and the computing device is configured to repeat a unit transformation method N number of times, the unit transformation method being a method to transform the generated source image (xₛ) into a harmonized image. In n^{th} iteration (n=1, . . . , N) of the unit transformation method, the computing device is configured to execute steps of: generating a corrected image corrected from a prepared n^{th} image using a predetermined reversible generative model; calculating a differential value of a distance between the corrected image and a source image; and generating a harmonized image by subtracting the differential value from the corrected image. The generating the corrected image comprises: generating a predetermined array by inputting the n^{th} image into the reversible generative model in a forward direction of the reversible generative model; and generating the corrected image by inputting a scaled array in a reverse direction of the reversible generative model, the scaled array being obtained by scaling a value of each element of the generated array by a predetermined scaling factor (1−α) (0<α<1).

According to the present invention, it is possible to provide a technology for configuring a harmonization network using only target domain data without source domain data during learning, and harmonizing data of the source domain only with the harmonization network. In other words, it is possible to provide a technology for transforming images of unlearned source domains into a harmonized image suitable for the target domain.

According to the present invention, in order to input source domain data into a predetermined network trained with target domain data, a harmonization network for transforming the source domain data into harmonized data can be trained using only target domain data.

According to the present invention, a flow model is trained only with target domain data, and the harmonized image is optimized by leveraging the invertibility of the flow model.

### BRIEF DESCRIPTION OF THE DRAWINGS

FIG. 1 shows the concept of a harmonization method provided according to the prior art.

FIG. 2 shows the concept of a harmonization method provided according to an embodiment of the present invention.

FIG. 3 shows the input-output relationship of the harmonization model used according to an embodiment of the present invention.

FIG. 4 is a flowchart showing a harmonized image generation method for generating a harmonized image for a predetermined target domain from a given source domain image, according to an embodiment of the present invention.

FIG. 5 is a figure to explain the unit transformation method shown in FIG. 4.

FIG. 6 is a flowchart showing a method for generating a harmonized image provided according to an embodiment of the present invention.

FIG. 7A, FIG. 7B, and FIG. 7C show an example of a business environment in which the harmonized image generation method provided according to an embodiment of the present invention is used.

### DETAILED DESCRIPTION OF EMBODIMENTS

Embodiments of the present invention will be described with reference to the accompanying drawings. However, the present invention is not limited to the embodiments described herein, and may be implemented in various different forms. The terminology used herein is not for limiting the scope of the present invention but for describing the embodiments. Furthermore, the singular forms used herein include the plural forms as well, unless otherwise indicated.

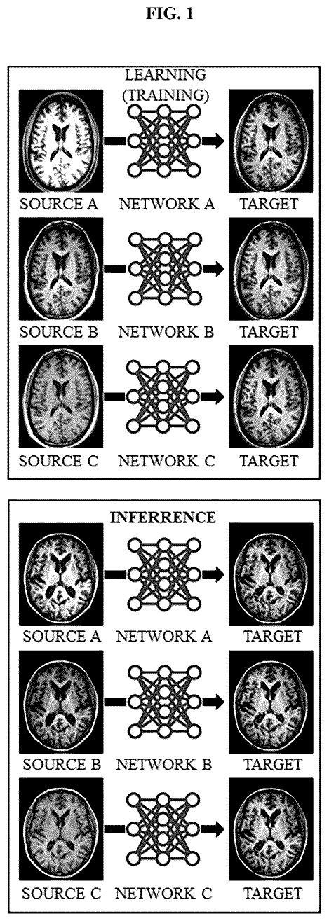

FIG. 1 shows the concept of a harmonization method provided according to the prior art.

The upper part of FIG. 1 shows learning processes or training processes of preparing each network for each source domain, which is used to harmonize an image of each source domain to a specific target domain.

The lower part of FIG. 1 shows processes of harmonizing an image of the source domain to the specific target domain using each network prepared in the upper part.

To describe the learning process (=training process) in the upper part of FIG. 1 in more detail, the network A, which is used to harmonize images of the source domain A to the specific target domain, is learned (=trained) using images belonging to the source domain A. Likewise, the network B (or C) used to harmonize the images of the source domain B (or C) to the specific target domain is learned using images belonging to the source domain B (or C).

To explain the inference process of the lower part of FIG. 1 in more detail, when an image A of the source domain A is input to the learned network A, the learned network A will generate a harmonized image for the target domain from the image A. Similarly, when an image B (or C) of the source domain B (or C) is input to the learned network B (or C), the learned network B (or C) will generate a harmonized image from the image B (or C) for the target domain.

If it is required to create another harmonized image for another target domain which is different from the above said target domain, it is necessary to train another network which is different from the above said network A, network B, and network C, and then use the trained another network. In other words, a corresponding network must be learned and prepared for each different combination of source domain and target domain.

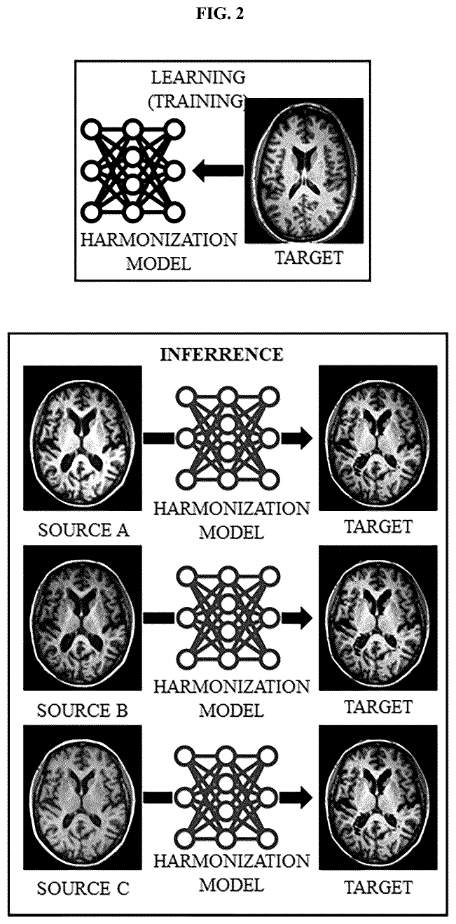

FIG. 2 shows the concept of a harmonization method provided according to an embodiment of the present invention.

The upper part of FIG. 2 shows the learning process of preparing a harmonization model (network) used to harmonize images of each source domain to a specific target domain using only data from the target domain, regardless of the source domain.

The lower part of FIG. 2 shows a process of harmonizing images from different source domains to the specific target domain using the harmonization model prepared in the upper part of FIG. 2. The said harmonization model is prepared for the specific target domain.

According to one embodiment of the present invention, different harmonization models must be prepared for different target domains. In other words, a harmonization model must be trained separately for each target domain. Therefore, if N different target domains are defined, in order to obtain harmonized images for each target domain, N different harmonization models must be completely learned and prepared.

However, according to one embodiment of the present invention, the learning process of each harmonization model is not influenced by data belonging to any source domain input to the harmonization model.

According to the prior art of FIG. 1, a corresponding network must be learned and prepared for each different combination of the source domain and the target domain.

In contrast, according to the present invention of FIG. 2, a network corresponding to the target domain can be learned and prepared regardless of the source domain. A corresponding network must be learned and prepared for each target domain.

For example, when N source domains and M target domains are defined, in order to harmonize the images of each source domain to each target domain, a total of N*M different networks must be prepared according to the prior art of FIG. 1.

In contrast, when N source domains and M target domains are defined, in order to harmonize the images of each source domain to each target domain, only a total of M different networks need to be prepared according to the present invention of FIG. 2. This is a technical advantage provided by the present invention.

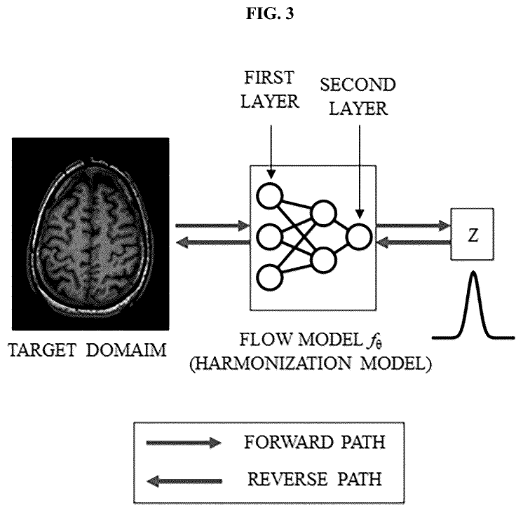

FIG. 3 shows the input-output relationship of the harmonization model used according to an embodiment of the present invention.

The harmonization model used according to one embodiment of the present invention may be a flow model (f_{θ}).

The flow model may be a reversible generative model.

The flow model may include a neural network.

When image data is input to the first layer of the flow model, the second layer of the flow model can output an array (Z) related to the probabilistic distribution of the image data (forward path). The output array (Z) of the probability distribution can be regarded as a prior distribution of the target domain. In the forward path, the first layer and the second layer may function as an input layer and an output layer, respectively, of the flow model.

Conversely, if an array (Z) related to the probability distribution is input to the second layer of the flow model, the first layer of the flow model can generate a new image that is the same as or similar to the image data (reverse path). In the reverse path, the first layer and the second layer may function as an output layer and an input layer, respectively, of the flow model.

In a preferred embodiment of the present invention, the flow model can be learned only with data belonging to a given target domain. At this time, learning image data belonging to the target domain may be input to the first layer of the flow model. At this time, the flow model can be learned in a way that the output array outputted by the second layer of the flow model follows a well-known distribution, for example, a Gaussian distribution.

In another embodiment of the present invention, the flow model can be learned only with data belonging to a given target domain. At this time, learning image data belonging to the target domain may be input to the first layer of the flow model. The output array outputted by the second layer of the flow model may be compared with a label array representing the distribution of the training image data. The flow model may be supervised to minimize the distance between the output array and the label array. The label array representing the distribution of the training image data may be an array that follows a Gaussian distribution.

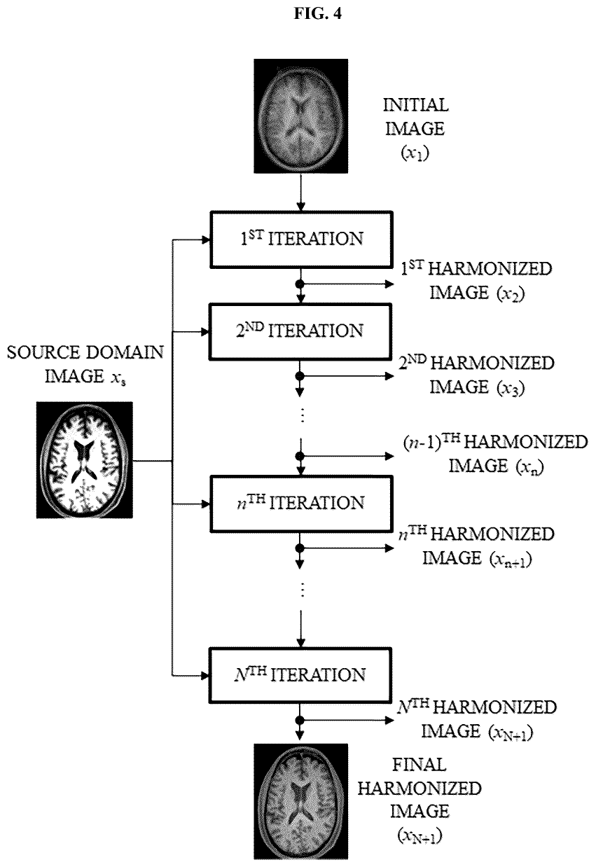

FIG. 4 is a flowchart showing a harmonized image generation method for generating a harmonized image for a predetermined target domain from a given source domain image, according to an embodiment of the present invention.

The harmonized image generation method may be configured to execute a unit transformation method for transforming the source image (xₛ) into a harmonized image a plurality of times, for example, N times. The n^{th} iteration shown in FIG. 4 represents the n^{th} execution of the unit transformation method.

Each time the unit transformation method is executed, a harmonized image for the target domain is generated from the source image (xₛ). As the number of executions of the unit transformation method increases, the accuracy of the harmonized image improves.

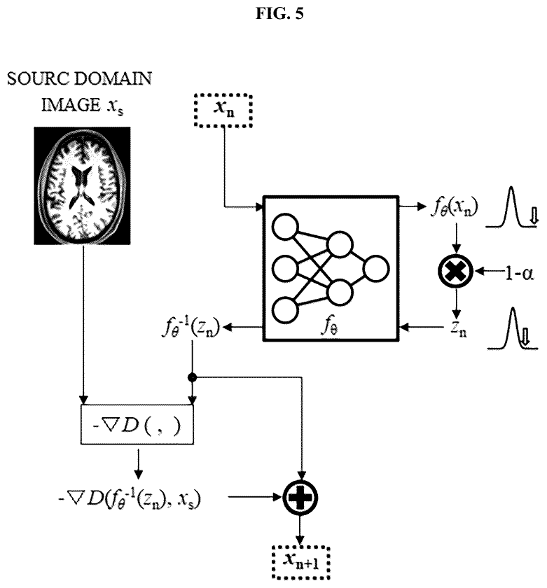

FIG. 5 is a figure to explain the unit transformation method shown in FIG. 4.

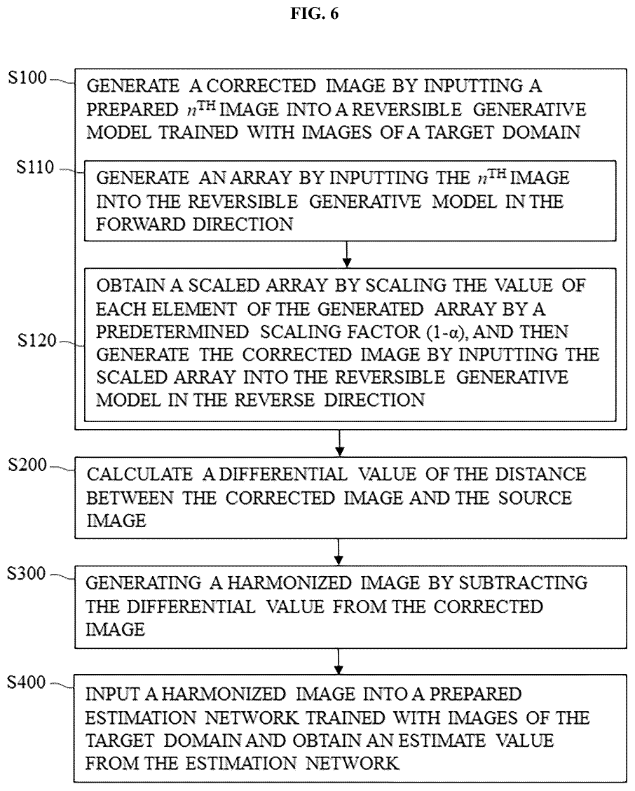

FIG. 6 is a flowchart showing a method for generating a harmonized image provided according to an embodiment of the present invention.

Hereinafter, it will be described with reference to FIG. 5 and FIG. 6 together.

In the n^{th} iteration of the unit transformation method (n=1, . . . , N), the unit transformation method may include the following steps.

In step S100, a computing device may generate a corrected image (f_{θ}⁻¹(zₙ)) by inputting a prepared n^{th} image (xₙ) into a predetermined reversible generative model (f_{θ}).

The predetermined reversible generative model (f_{θ}) may be a reversible generative model learned using only data of the target domain (=first domain) described in FIG. 3.

Here, the reversible generative model may be a flow model.

In step S200, the computing device may calculate a differential value (∇D(f_{θ}⁻¹(zₙ), xₛ)) of the distance between the corrected image (f_{θ}⁻¹(zₙ)) and the source image (xₛ).

In step S300, a harmonized image (xₙ₊₁) can be generated by subtracting the differential value (∇D(f_{θ}⁻¹(zₙ), xₛ)) from the corrected image (f_{θ}⁻¹(zₙ)).

At this time, step S100 may include steps S110 and S120 below.

In step S110, the computing device may generate an array (f_{θ}(xₙ)) by inputting the n^{th} image into the reversible generative model (f_{θ}) in the forward direction of the reversible generative model (f_{θ}).

In step S120, the computing device may obtain a scaled array (zₙ) by scaling the value of each element of the generated array (f_{θ}(xₙ)) by a predetermined scaling factor (1−α), and then generate the corrected image (f_{θ}⁻¹(zₙ)) by inputting the scaled array (zₙ) into the reversible generative model in the reverse direction of the reversible generative model (f_{θ}) (where 0<α<1).

At this time, the n^{th} image (xₙ) input to the reversible generative model (f_{θ}) at the n^{th} iteration of the unit transformation method may be the harmonized image (xₙ₋₁) generated in the (n−1)^{th} iteration of the unit transformation method (where n=2, . . . , N).

At this time, the reversible generative model (f_{θ}) may be a reversible generative model learned using only images belonging to the first domain. Here, the first domain is the target domain.

At this time, the first image (x₁) input to the reversible generative model (f) in the first iteration (n=1) of the unit transformation method is a random image, or one of the images belonging to the first domain, or a representative image of images belonging to the first domain. The representative image may be an average image of images belonging to the first domain.

At this time, the source image (xₛ) may be an image belonging to a second domain different from the first domain. At this time, the second domain is a source domain.

At this time, the first domain may be a domain composed of images output by a first MR scanner, and the second domain may be a domain composed of images output by a second MR scanner.

According to an embodiment of the present invention, an MR image processing method using a predetermined estimation network learned using images belonging to a target domain can be provided. The MR image processing method may include the above said steps S100, S200, and S300 described in FIG. 6, and step S400 described later.

The said steps S100, S200, and S300 are the same as described above.

In step S400, the computing device may input a harmonized image (x_{N+1}) into a predetermined estimation network and obtain an estimate value from the estimation network. Here, the harmonized image (x_{N+1}) is an image which has been generated by the unit transformation method executed at the N^{th} iteration. Here, the predetermined estimation network is an estimation network which has been trained using images belonging to the first domain (target domain).

The predetermined estimation network may be any kind of estimation network.

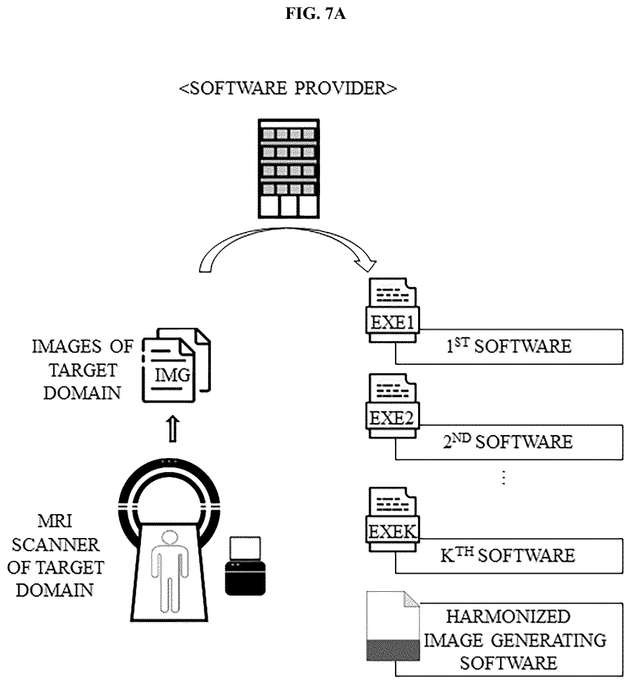

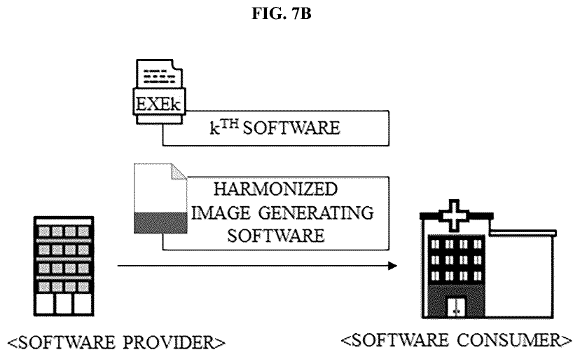

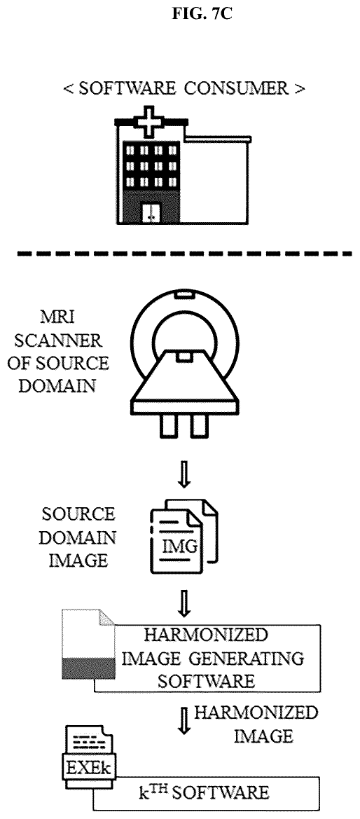

FIG. 7A, FIG. 7B, and FIG. 7C show an example of a business environment in which the harmonized image generation method provided according to an embodiment of the present invention is used.

Explaining with reference to FIG. 7A, a certain software provider uses target domain images generated using an MRI scanner of a target domain (=first domain) to produce k^{th} software (k=1, . . . , K). The k^{th} software may be software that receives an image and processes the received image.

Additionally, the software provider may produce harmonized image generation software, which is software that executes an MR image processing method provided according to an embodiment of the present invention, illustrated in FIG. 5. The harmonized image generation software may also be produced using target domain images generated using the MRI scanner of the target domain.

Explaining with reference to FIG. 7B, the software provider may supply the k^{th} software and the harmonized image generation software to software consumers.

Explaining with reference to FIG. 7C, the software consumers can prepare a source domain image generated using an MRI scanner of a source domain (=second domain). And, a harmonized image can be generated from the source domain image using the harmonized image generation software. Then, the generated harmonized image may be input into the k^{th} software, and a required results can be obtained from the k^{th} software processing the generated harmonized image.

Those skilled in the art could easily make various alterations or modifications to the above-mentioned embodiments of the present invention without departing the essential characteristics of the present invention. The claims that do not refer to each other may be combined with each other within the scope of understanding of the present disclosure.

### ACKNOWLEDGMENT

The present invention was developed in the process of carrying out research projects following:
Research project No. 1: National research and development project that supported this invention—[Assignment number]1711188595, [Assignment number]2022R1A4A1030579, [Ministry Name] Ministry of Science and ICT, [Name of project management (professional) organization] National Research Foundation of Korea, [Research Project Name] Basic Laboratory Support Project, [Research project name] Deep-designed medical imaging basic laboratory, [Contribution rate]50/100, [Name of project carrying out organization] Seoul National University Industry-Academic Cooperation Foundation, [Research period]2022.06.01˜2025.02.28.
Research project No. 2: National research and development project that supported this invention—[Assignment number]1615013209, [Assignment number] Grant 21NPSS-C163415-01, [Name of Ministry] Ministry of Land, Infrastructure and Transport, [Name of project management (professional) organization] Korea Agency for Land, Infrastructure and Transport Science and Technology Promotion, [Research project name] Development of next-generation interpersonal security search technology, [Research project title] Development of next-generation interpersonal security search technology, [Contribution rate]50/100, [Name of project carrying out organization] Seoul National University Industry-Academic Cooperation Foundation, [Research period]2021.04.01˜2023.12.31.

What is claimed is:

1. An MR image processing method that repeats, by a computing device, a unit transformation method N number of times, wherein,
the unit transformation method in n^{th} iteration comprises:
generating, by the computing device, a corrected image corrected from a prepared n^{th} image using a predetermined reversible generative model;
calculating, by the computing device, a differential value of a distance between the corrected image and a source image; and
generating, by the computing device, a harmonized image by subtracting the differential value from the corrected image;
wherein, the generating the corrected image comprises: generating a predetermined array by inputting the n^{th} image into the reversible generative model in a forward direction of the reversible generative model; and generating the corrected image by inputting a scaled array in a reverse direction of the reversible generative model, the scaled array being obtained by scaling a value of each element of the generated array by a predetermined scaling factor (1−α) (0<α<1).

2. The method of claim 1, wherein the reversible generative model is a prior model.

3. The method of claim 1, wherein the n^{th} image (xₙ) input to the reversible generative model (f_{θ}) in the n^{th} iteration of the unit transformation method is the harmonized image (xₙ) generated in (n−1)^{th} iteration of the unit transformation method (n=2, . . . , N).

4. The method of claim 1, wherein,
the reversible generative model (f_{θ}) is learned using only images belonging to a first domain,
in a first iteration (n=1) of the unit transformation method, a first image (x₁) input to the reversible generative model (f_{θ}) is either a random image or one of images belonging to the first domain, or a representative image of images belonging to the first domain.

5. The method of claim 1, wherein,
the reversible generative model (f_{θ}) is learned using only images belonging to a first domain, and
the source image (xₛ) is an image belonging to a second domain different from the first domain.

6. The method of claim 1, further comprising:
inputting, by the computing device, a harmonized image (x_{N+1}) generated by the unit transformation method performed by the n^{th} iteration into a predetermined estimation network learned using images belonging to the first domain; and
obtaining, by the computing device, an estimate from the estimation network according to the inputted harmonized image (x_{N+1}).

7. The method of claim 6, wherein,
the source image is a first MRI image of a brain, and
the estimation network is a network that outputs a location of a tumor among the first MRI image of the brain when the first MRI image of the brain is input to the estimation network.

8. The method of claim 6, wherein,
the source image is an MRI image of a human body, and
the estimation network is a network that outputs a location of a lesion in the MRI image of the human body when the MRI image of the human body is input to the estimation network.

9. The method of claim 5, wherein,
the first domain is a domain composed of images output by a first MR scanner, and
the second domain is a domain composed of images output by a second MR scanner.

10. The method of claim 1, wherein,
the reversible generative model (f_{θ}) is learned using only images belonging to a first domain,
a method for learning the reversible generative model (f_{θ}) comprises:
inputting a selected image belonging to the first domain into the reversible generative model in the forward direction of the reversible generative model to generate an array from the reversible generative model; and
changing parameters of the reversible generative model to reduce a difference between distributions of images belonging to the first domain and the generated array.

11. An MR image processing method, comprising:
generating, by an MRI scanner, a source image (xₛ); and
repeating, by a computing device, a unit transformation method N number of times, the unit transformation method being a method to transform the generated source image (xₛ) into a harmonized image,
wherein,
the unit transformation method in n^{th} iteration comprises:
generating, by the computing device, a corrected image corrected from a prepared n^{th} image using a predetermined reversible generative model;
calculating, by the computing device, a differential value of a distance between the corrected image and a source image; and
generating, by the computing device, a harmonized image by subtracting the differential value from the corrected image,
wherein, the generating the corrected image comprises: generating a predetermined array by inputting the n^{th} image into the reversible generative model in a forward direction of the reversible generative model; and generating the corrected image by inputting a scaled array in a reverse direction of the reversible generative model, the scaled array being obtained by scaling a value of each element of the generated array by a predetermined scaling factor (1−α) (0<α<1).

12. An MR image processing system, comprising:
an MRI scanner; and
a computing device,
wherein,
the MRI scanner is configured to scan a scan object to generate a source image (xₛ),
the computing device is configured to repeat a unit transformation method N number of times, the unit transformation method being a method to transform the generated source image (xₛ) into a harmonized image,
in n^{th} iteration (n=1, . . . , N) of the unit transformation method, the computing device is configured to execute steps of: generating a corrected image corrected from a prepared n^{th} image using a predetermined reversible generative model; calculating a differential value of a distance between the corrected image and a source image; and generating a harmonized image by subtracting the differential value from the corrected image;
wherein, the generating the corrected image comprises: generating a predetermined array by inputting the n^{th} image into the reversible generative model in a forward direction of the reversible generative model; and generating the corrected image by inputting a scaled array in a reverse direction of the reversible generative model, the scaled array being obtained by scaling a value of each element of the generated array by a predetermined scaling factor (1−α) (0<α<1).
